(19) United States
(12) Patent Application Publication
(10) Pub. No.: US 2024/0390237 A1
(43) Pub. Date: Nov. 28, 2024

(54) SOLID COSMETIC

(71) Applicant: Shiseido Company, Ltd., Tokyo (JP)

(72) Inventors: Shun KUBOTA, Tokyo (JP); Kosuke ARIMOTO, Tokyo (JP)

(73) Assignee: Shiseido Company, Ltd., Tokyo (JP)

(21) Appl. No.: 18/037,730

(22) PCT Filed: Dec. 20, 2021
(86) PCT No.: PCT/JP2021/046946

§ 371 Date: Oct. 20, 2023

(30) Foreign Application Priority Data
Dec. 22, 2020 (JP) 2020-212031

(51) Int. Cl. A61K 8/02; A61Q 1/06; A61Q 1/10; A61Q 19/00

(52) U.S. Cl. CPC A61K 8/0216; A61Q 1/06; A61Q 1/10; A61Q 19/007; A61K 2800/592

(57) ABSTRACT

Provided is a composition solidified using a solidifying agent that is dissolved in a single-phase composition, which has been obtained by making a moisturizer with an expected moisturizing effect compatible with an oil phase having an occlusion effect.

A solid composition comprising:
an oily liquid medium that separates when mixed with water at 1:1;
an aqueous liquid medium that is an organic liquid medium other than water, that forms a homogeneous phase when mixed with water at 1:1, and that forms a homogeneous phase when mixed with the oily liquid medium; and
an aqueous solidifying agent that is soluble in the aqueous liquid medium and that gels and/or solidifies the aqueous liquid medium, and/or an oily solidifying agent that is soluble in the oily liquid medium and that gels and/or solidifies the oily liquid medium,
wherein the oily liquid medium and aqueous liquid medium are gelled and/or solidified with the aqueous solidifying agent and/or oily solidifying agent, and
the total amount of the aqueous liquid medium, oily liquid medium, aqueous solidifying agent, and oily solidifying agent is 90% by mass or more of the amount of the composition excluding powder.

### RELATED APPLICATION

The present application claims priority from Japanese Patent Application No. 2020-212031 filed on Dec. 22, 2020, which is incorporated herein.

### FIELD OF THE INVENTION

The present invention relates to a solid cosmetic, and particularly relates to a cosmetic solidified with a solidifying agent.

### BACKGROUND OF THE INVENTION

Conventionally, when solid cosmetics such as lipsticks are produced, solid oils such as waxes and hardened oils, or oily gelling agents such as 12-hydroxystearic acid, are generally used (Patent Literatures 1 to 3).

### CITATION LIST

### Patent Literature

Patent Literature 1: Japanese Patent No. 6465700
Patent Literature 2: Japanese Patent No. 6468891
Patent Literature 3: Japanese Patent No. 6465701

### DISCLOSURE OF THE INVENTION

### Problem to be Solved by the Invention

However, solid cosmetics solidified using the solid oils or oily gelling agents cannot be solidified with moisturizers dissolved therein, as typified by so-called solid lipsticks. On the other hand, it is easy to blend moisturizers in water-soluble solids; however, it is not possible to achieve solidification while dissolving oil contents that have an occlusion effect.

The present invention has been made in view of the conventional art, and its problem to be solved is to provide a cosmetic solidified using a solidifying agent that is dissolved in a single-phase composition, which has been obtained by making a moisturizer with an expected moisturizing effect compatible with an oil phase having an occlusion effect.

### Means to Solve the Problem

Specifically, the present invention includes the following contents.
(1) A solid composition comprises:
an oily liquid medium that separates when mixed with water at 1:1;
an aqueous liquid medium that is an organic liquid medium other than water, that forms a homogeneous phase when mixed with water at 1:1, and that forms a homogeneous phase when mixed with the oily liquid medium; and
an aqueous solidifying agent that is soluble in the aqueous liquid medium and that gels and/or solidifies the aqueous liquid medium, and/or an oily solidifying agent that is soluble in the oily liquid medium and that gels and/or solidifies the oily liquid medium,
wherein the oily liquid medium and aqueous liquid medium are gelled and/or solidified with the aqueous solidifying agent and/or oily solidifying agent, and
the total amount of the aqueous liquid medium, oily liquid medium, aqueous solidifying agent, and oily solidifying agent is 90% by mass or more of the amount of the composition excluding powder.
(2) The solid composition comprises an oil-absorbing powder containing an oil content other than the oily liquid medium.
(3) The solid composition has a hardness of 10 to 500 when a probe with a diameter of 5.6 mm penetrates at 20 mm/min using a rheometer.
(4) In the solid composition,
the oily liquid medium is one or two or more selected from the group consisting of, as phenyl-containing substances, cinnamic acid derivatives selected from ethylhexyl methoxycinnamate, isopropyl methoxycinnamate, and isoamyl methoxycinnamate; PABA derivatives selected from para-aminobenzoic acid, ethyl PABA, ethyl-dihydroxypropyl PABA, ethylhexyl-dimethyl PABA, and glyceryl PABA; salicylic acid derivatives selected from homosalate, ethylhexyl salicylate, dipropylene glycol salicylate, and TEA salicylate; benzophenone derivatives selected from benzophenone-1, benzophenone-2, benzophenone-3 or oxybenzone, benzophenone-4, benzophenone-5, benzophenone-6, benzophenone-8, benzophenone-9, and benzophenone-12; benzylidene camphor derivatives selected from 3-benzylidene camphor, 4-methylbenzylidene camphor, benzylidene camphor sulfonic acid, benzalkonium methosulfate camphor, terephthalylidene dicamphor sulfonic acid, and polyacrylamide methylbenzylidene camphor; triazine derivatives selected from anisotriazine, ethylhexyltriazone, diethylhexylbutamidotriazone, 2,4,6-tris(diisobutyl-4′-aminobenzalmalonate)-s-triazine, 2,4-bis-[{4-(2-ethylhexyloxy)-2-hydroxy}-phenyl]-6-(4-methoxyphenyl)-1,3,5-triazine, and 2,4,6-tris [4-(2-ethylhexyloxycarbonyl) anilino]1,3,5-triazine; phenylbenzimidazole derivatives selected from disodium phenyl dibenzimidazole tetrasulfonate; phenylbenzotriazole derivatives selected from drometrizole trisiloxane and methylene bis(benzotriazolyl tetramethylbutylphenol); anthranil derivatives selected from menthyl anthranilate; imidazoline derivatives selected from ethylhexyl dimethoxy benzylidene dioxoimidazoline propionate; benzalmalonate derivatives selected from polyorganosiloxanes with benzalmalonate functional groups; 4,4-diarylbutadiene derivatives selected from 1,1-dicarboxy (2,2′-dimethylpropyl)-4,4-diphenylbutadiene; hexyl diethylaminohydroxybenzoylbenzoate, and bis-ethylhexyloxyphenol methoxyphenyl triazine-; and diols and triols that are incompatible with water at 1:1;
the aqueous liquid medium is one or two or more selected from the group consisting of dipropylene glycol, polyethylene glycols that are liquid at room temperature, PEG/PPG-14/7 dimethyl ether, PEG/PPG-9/2 dimethyl ether, PEG/PPG-36/41 dimethyl ether, PEG/PPG-55/28 dimethyl ether, PEG/PPG-17/4 dimethyl ether, 1,2-hexanediol, isopropylene glycol, propylene glycol, and 1,3-butylene glycol;
the aqueous solidifying agent is one or two or more selected from the group consisting of bis-polyethylene glycol methyl ether dimethylsilane, polyethylene glycol·decyltetradeceth-20·hexamethylene diisocyanate copolymer, polyethylene glycols that are solid at 25° C., and polyurethane-59; and
the oily solidifying agent is one or two or more selected from the group consisting of dibutyl lauroyl glutamide, dibutyl ethylhexanoyl glutamide, polyamide-3, polyamide-8, and 12-hydroxystearic acid.

### Effect of the Invention

In the present invention, an oil-soluble medium and a water-soluble medium are used to solidify a homogeneous single-phase liquid composition using oily and/or aqueous solidifying agents, whereby it is possible to obtain various physical properties.

### BEST MODE FOR CARRYING OUT THE INVENTION

Preferred embodiments of the present invention will be described below.

The present invention is intended to solidify an oily composition using an oily liquid medium, an aqueous liquid medium that is compatible with the oily liquid medium, and various solidifying agents.

### [Oily Liquid Medium]

In the present invention, the oily liquid medium refers to an oily substance that separates when mixed with water at a mass ratio of 1:1.

Specific examples include ethylhexyl salicylate, cinnamic acid derivatives, such as ethylhexyl methoxycinnamate, isopropyl methoxycinnamate, and isoamyl methoxycinnamate; PABA derivatives, such as para-aminobenzoic acid (hereinafter abbreviated as “PABA”), ethyl PABA, ethyl-dihydroxypropyl PABA, ethylhexyl-dimethyl PABA, and glyceryl PABA; salicylic acid derivatives, such as homosalate, ethylhexyl salicylate, dipropylene glycol salicylate, and TEA salicylate; benzophenone derivatives, such as benzophenone-1, benzophenone-2, benzophenone-3 or oxybenzone, benzophenone-4, benzophenone-5, benzophenone-6, benzophenone-8, benzophenone-9, and benzophenone-12; benzylidene camphor derivatives, such as 3-benzylidene camphor, 4-methylbenzylidene camphor, benzylidene camphor sulfonic acid, benzalkonium methosulfate camphor, terephthalylidene dicamphor sulfonic acid, and polyacrylamide methylbenzylidene camphor; triazine derivatives, such as anisotriazine, ethylhexyltriazone, diethylhexylbutamidotriazone, 2,4,6-tris(diisobutyl-4′-aminobenzalmalonate)-s-triazine, 2,4-bis-[{4-(2-ethylhexyloxy)-2-hydroxy}-phenyl]-6-(4-methoxyphenyl)-1,3,5-triazine, and 2,4,6-tris [4-(2-ethylhexyloxycarbonyl) anilino]1,3,5-triazine; phenylbenzimidazole derivatives, such as disodium phenyl dibenzimidazole tetrasulfonate; phenylbenzotriazole derivatives, such as drometrizole trisiloxane and methylene bis(benzotriazolyl tetramethylbutylphenol); anthranil derivatives, such as menthyl anthranilate; imidazoline derivatives, such as ethylhexyl dimethoxy benzylidene dioxoimidazoline propionate; benzalmalonate derivatives, such as polyorganosiloxanes with benzalmalonate functional groups; 4,4-diarylbutadiene derivatives, such as 1,1-dicarboxy (2,2′-dimethylpropyl)-4,4-diphenylbutadiene; hexyl diethylaminohydroxybenzoylbenzoate, and bis-ethylhexyloxyphenol methoxyphenyl triazine.

Examples of particularly preferably used oily liquid media include ethylhexyl salicylate and ethylhexyl methoxycinnamate.

In relation to an aqueous liquid medium described later, a suitable blending amount of the oily liquid medium is such that the mass ratio of oily liquid medium/(aqueous liquid medium+oily liquid medium) is preferably in the range of 0.1 to 0.9, and more preferably 0.3 to 0.7.

In the present invention, the “oily liquid medium” may be an oil content that is singly solid at room temperature (20° C.), but is dissolved in other coexisting oily liquid media and be a liquid as a whole.

### [Aqueous Liquid Medium]

In the present invention, the aqueous liquid medium refers to an organic liquid medium other than water, and refers to an aqueous substance that forms a homogeneous phase when mixed with water at 1:1 and that forms a homogeneous phase when mixed with the oily liquid medium.

Specific examples include dipropylene glycol, polyethylene glycols that are liquid at room temperature (e.g., PEG-8), alkylene oxide derivatives, 1,2-hexanediol, isopropylene glycol, propylene glycol, and 1,3-butylene glycol.

More preferred are dipropylene glycol, polyethylene glycols that are liquid at room temperature (e.g., PEG-8), alkylene oxide derivatives, and 1,2-hexanediol.

In some examples, 1,3-propanediol, diglycerol, and dynamite glycerol can be included.

Particularly preferred are dipropylene glycol and polyethylene glycols that are liquid at room temperature.

### [Aqueous Solidifying Agent]

Aqueous solidifying agents that can be particularly preferably used in the present invention are soluble in the aqueous liquid media and have the function of solidifying the aqueous liquid media.

Specific examples include bis-polyethylene glycol methyl ether dimethylsilane, polyethylene glycols that are solid at 25° C. (e.g., polyethylene glycol-400), polyethylene glycol·decyltetradeceth-20·hexamethylene diisocyanate copolymer, and the like.

### [Oily Solidifying Agent]

In the present invention, a small amount of oily solidifying agent can be added, although it is not an essential component.

Examples of oily solidifying agents preferably used in the present invention include 12-hydroxystearic acid, dibutyl lauroyl glutamide, and the like.

### [Blending Amount of Each Component]

In the present invention, the total amount of the aqueous liquid medium, oily liquid medium, and aqueous solidifying agent is preferably 90% by mass or more in the composition excluding powder.

If the total amount of the essential components is less than 90% by mass in the composition excluding powder, the solidification properties may be reduced. The powder mentioned herein also includes porous powders that support oily components and aqueous components. Since these powders do not affect the affinity of the aqueous liquid medium and oily liquid medium, the powders, including the supported oily components and aqueous components, may not be taken into consideration from the system.

Moreover, in the present invention, the mass ratio of oily liquid medium/(aqueous liquid medium+oily liquid medium) is preferably 0.1 to 0.9, and more preferably 0.3 to 0.7. If the mass ratio of the oily liquid medium is less than 0.3, it is difficult to demonstrate the characteristics as an oily composition. If the mass ratio exceeds 0.7, the proportion of the aqueous liquid medium relatively decreases, and the solidification properties tend to deteriorate.

### [Other Components]

Cosmetics using the cosmetic raw materials according to the present invention may be blended with, as required, other components, such as inorganic powders, organic powders, esters, anionic surfactants, cationic surfactants, amphoteric surfactants, nonionic surfactants, moisturizers, water-soluble polymers, thickeners, coating agents, UV absorbers, sequestrants, lower alcohols, polyhydric alcohols, sugars, amino acids, organic amines, polymer emulsions, pH adjusters, skin nutrients, vitamins, antioxidants, antioxidant aids, fragrances, and water, within the range in which the effects of the present invention are not impaired. Such cosmetics can be produced by conventional methods depending on the desired dosage forms.

Examples of inorganic powders include talc, boron nitride, sericite, natural mica, calcined mica, synthetic mica, synthetic sericite, alumina, mica, kaolin, bentonite, smectite, calcium carbonate, magnesium carbonate, calcium phosphate, silicic anhydride, magnesium oxide, tin oxide, iron oxide, yttrium oxide, chromium oxide, zinc oxide, cerium oxide, aluminum oxide, magnesium oxide, chromium hydroxide, iron blue, ultramarine blue, calcium phosphate, aluminum hydroxide, barium sulfate, magnesium sulfate, silicic acid, magnesium aluminum silicate, calcium silicate, barium silicate, magnesium silicate, aluminum silicate, strontium silicate, silicon carbide, magnesium fluoride, tungstic acid metal salts, magnesium aluminate, magnesium aluminometasilicate, chlorohydroxyaluminum, clay, zeolite, hydroxyapatite, ceramic powder, spinel, mullite, cordierite, aluminum nitride, titanium nitride, silicon nitride, lanthanum, samarium, tantalum, terbium, europium, neodymium, Mn—Zn ferrite, Ni—Zn ferrite, silicon carbide, cobalt titanate, barium titanate, iron titanate, lithium cobalt titanate, cobalt aluminate, antimony-containing tin oxide, tin-containing indium oxide, magnetite, aluminum powder, gold powder, silver powder, platinum powder, copper powder, precious metal colloid, iron powder, zinc powder, cobalt blue, cobalt violet, cobalt green, low-order titanium oxide, fine titanium oxide, butterfly-like barium sulfate, petal-like zinc oxide, tetrapod-like zinc oxide, and fine zinc oxide; as pearl pigments, titanium oxide-coated isinglass, titanium oxide-coated mica, titanium oxide-coated synthetic mica, titanium oxide-coated silica, titanium oxide-coated talc, zinc oxide-coated silica, titanium oxide-coated colored mica, red iron oxide-coated mica titanium, red iron oxide/black iron oxide-coated mica titanium, carmine-coated mica titanium, iron blue-coated mica titanium, and the like.

Examples of organic powders include (e.g., silicone elastomer powder, silicone powder, silicone resin-coated silicone elastomer powder, polyamide resin powder (nylon powder), polyethylene powder, polymethyl methacrylate powder (e.g. methyl methacrylate crosspolymer), polystyrene powder, styrene-acrylic acid copolymer resin powder, benzoguanamine resin powder, polytetrafluoroethylene powder, and cellulose powder); organic pigments, such as zirconium, barium, or aluminum lake (e.g., organic pigments, such as Red No. 201 (INCI Name: CI 15850, Red 6), Red No. 202 (INCI Name: CI 15850, Red 7), Red No. 204 (INCI Name: CI 15585, Red 9), Red No. 205 (INCI Name: CI 15630, Red 10), Red No. 220 (INCI Name: CI 15880, Red 34), Red No. 226 (INCI Name: CI 73360, Red 30), Red No. 228 (INCI Name: CI 12085, Red 36), Red No. 405 (INCI Name: CI 15865), Orange No. 203 (INCI Name: CI 12075, Orange 17), Orange No. 204 (INCI Name: CI 21110), Yellow No. 205 (INCI Name: CI 21090), Yellow No. 401 (INCI Name: CI 11680, Yellow 5), and Blue No. 404 (INCI Name: CI 74160)), and the like.

Examples of anionic surfactants include fatty acid soaps (e.g., sodium laurate and sodium palmitate); higher alkyl sulfuric acid ester salts (e.g., sodium lauryl sulfate and potassium lauryl sulfate); alkyl ether sulfuric acid ester salts (e.g., triethanolamine POE-lauryl sulfate and sodium POE-lauryl sulfate); N-acyl sarcosine acids (e.g., sodium lauroyl sarcosinate); higher fatty acid amide sulfonates (e.g., sodium N-myristoyl-N-methyl taurate, sodium coconut fatty acid methyl taurine, and sodium lauryl methyl taurine); phosphoric acid ester salts (sodium POE-oleyl ether phosphate, POE-stearyl ether phosphate, etc.); sulfosuccinates (e.g., sodium di-2-ethylhexyl sulfosuccinate, sodium monolauroyl monoethanolamide polyoxyethylene sulfosuccinate, and sodium lauryl polypropylene glycol sulfosuccinate); alkylbenzene sulfonates (e.g., sodium linear dodecylbenzenesulfonate, triethanolamine linear dodecylbenzenesulfonate, and linear dodecylbenzenesulfonic acid); higher fatty acid ester sulfuric acid ester salts (e.g., sodium hydrogenated coconut oil fatty acid glycerol sulfate); N-acyl glutamates (e.g., monosodium N-lauroyl glutamate, disodium N-stearoyl glutamate, and monosodium N-myristoyl-L-glutamate); sulfated oils (e.g., Turkey red oil); POE-alkyl ether carboxylic acids; POE-alkylallyl ether carboxylates; α-olefin sulfonates; higher fatty acid ester sulfonates; secondary alcohol sulfates; higher fatty acid alkylolamide sulfates; sodium lauroyl monoethanolamide succinate; ditriethanolamine N-palmitoyl aspartate; sodium caseinate; and the like.

Examples of cationic surfactants include alkyltrimethylammonium salts (e.g., stearyltrimethylammonium chloride and lauryltrimethylammonium chloride); alkylpyridinium salts (e.g., cetylpyridinium chloride); distearyl dimethyl ammonium chloride, dialkyldimethylammonium salts; poly(N,N′-dimethyl-3,5-methylenepiperidinium) chloride; alkyl quaternary ammonium salts; alkyldimethylbenzylammonium salts; alkylisoquinolinium salts; dialkyl morphonium salts; POE-alkylamines; alkylamine salts; polyamine fatty acid derivatives; amyl alcohol fatty acid derivatives; benzalkonium chloride; benzethonium chloride; and the like.

Examples of amphoteric surfactants include imidazoline-based amphoteric surfactants (e.g., sodium 2-undecyl-N,N,N-(hydroxyethyl carboxymethyl)-2-imidazoline and 2-cocoyl-2-imidazolinium hydroxide-1-carboxyethyloxy disodium salt); betaine-based surfactants (e.g., 2-heptadecyl-N-carboxymethyl-N-hydroxyethylimidazolinium betaine, betaine lauryldimethylaminoacetate, alkylbetaine, amidobetaine, and sulfobetaine); and the like.

Examples of lipophilic nonionic surfactants include sorbitan fatty acid esters (e.g., sorbitan monooleate, sorbitan monoisostearate, sorbitan monolaurate, sorbitan monopalmitate, sorbitan monostearate, sorbitan sesquioleate, sorbitan trioleate, diglycerol sorbitan penta-2-ethylhexylate, and diglycerol sorbitan tetra-2-ethylhexylate); glycerol polyglycerol fatty acids (e.g., mono-cottonseed fatty acid glycerol, glycerol monoerucate, glycerol sesquioleate, glycerol monostearate, glycerol α,α′-oleate pyroglutamate, and glycerol monostearate malate); propylene glycol fatty acid esters (e.g., propylene glycol monostearate); hydrogenated castor oil derivatives; glycerol alkyl ether; and the like.

Examples of hydrophilic nonionic surfactants include POE-sorbitan fatty acid esters (e.g., POE-sorbitan monooleate, POE-sorbitan monostearate, POE-sorbitan monooleate, and POE-sorbitan tetraoleate); POE-sorbit fatty acid esters (e.g., POE-sorbit monolaurate, POE-sorbit monooleate, POE-sorbit pentaoleate, and POE-sorbit monostearate); POE-glycerol fatty acid esters (e.g., POE-monooleates, such as POE-glycerol monostearate, POE-glycerol monoisostearate, and POE-glycerol triisostearate); POE-fatty acid esters (e.g., POE-distearate, POE-monodioleate, and ethylene glycol distearate); POE-alkyl ethers (e.g., POE-lauryl ether, POE-oleyl ether, POE-stearyl ether, POE-behenyl ether, POE-2-octyldodecyl ether, and POE-cholestanol ether); Pluronic (registered trademark) types (e.g., Pluronic (registered trademark)); POE·POP-alkyl ethers (e.g., POE·POP-cetyl ether, POE·POP-2-decyltetradecyl ether, POE·POP-monobutyl ether, POE·POP-hydrogenated lanolin, and POE·POP-glycerol ether); tetra-POE/tetra-POP-ethylenediamine condensates (e.g., Tetronic); POE-castor oil hydrogenated castor oil derivatives (e.g., POE-castor oil, POE-hydrogenated castor oil, POE-hydrogenated castor oil monoisostearate, POE-hydrogenated castor oil triisostearate, POE-hydrogenated castor oil monopyroglutamate monoisostearate diester, and POE-hydrogenated castor oil maleic acid); POE-beeswax·lanoline derivatives (e.g., POE-sorbit beeswax); alkanolamides (e.g., coconut oil fatty acid diethanolamide, lauric acid monoethanolamide, and fatty acid isopropanolamide); POE-propylene glycol fatty acid esters; POE-alkylamines; POE-fatty acid amides; sucrose fatty acid esters; alkylethoxydimethylamine oxides; trioleyl phosphates; and the like.

Examples of moisturizers include xylitol, sorbitol, maltitol, chondroitin sulfate, hyaluronic acid, mucoitin sulfate, caronic acid, atelocollagen, cholesteryl-12-hydroxystearate, sodium lactate, bile salts, dl-pyrrolidone carboxylate, alkylene oxide derivatives, short-chain soluble collagen, diglycerol (EO) PO adducts, Rosa roxburghii fruit extract, Achillea millefolium extract, melilot extract, and the like.

These moisturizers are dissolved in the aqueous liquid media characteristic in the present invention and thus may affect the affinity of the oily liquid media and aqueous liquid media. Accordingly, it is necessary to add these moisturizers while taking into consideration the affinity with the oily liquid media.

Examples of natural water-soluble polymers include plant-based polymers (e.g., gum arabic, gum tragacanth, galactan, guar gum, gum carob, gum karaya, carrageenan, pectin, agar, quince seed (quince), algecolloid (brown algae extract), starch (rice, corn, potato, and wheat), and glycyrrhizic acid); microbial polymers (e.g., xanthan gum, dextran, succinoglycan, and pullulan); animal-based polymers (e.g., collagen, casein, albumin, and gelatin); and the like.

Examples of semi-synthetic water-soluble polymers include starch-based polymers (e.g., carboxymethyl starch and methylhydroxypropyl starch); cellulose-based polymers (e.g., methylcellulose, ethylcellulose, methylhydroxypropylcellulose, hydroxyethylcellulose, sodium cellulose sulfate, hydroxypropylcellulose, carboxymethylcellulose, sodium carboxymethylcellulose, crystalline cellulose, and cellulose powder); alginic acid-based polymers (e.g., sodium alginate and alginic acid propylene glycol ester); and the like.

Examples of synthetic water-soluble polymers include vinyl-based polymers (e.g., polyvinyl alcohol, polyvinyl methyl ether, polyvinylpyrrolidone, and carboxyvinyl polymer); polyoxyethylene-based polymers (e.g., polyoxyethylene-polyoxypropylene copolymers, such as polyethylene glycols 20,000, 40,000, and 60,0000); acrylic polymers (e.g., sodium polyacrylate, polyethyl acrylate, and polyacrylamide); polyethylenimine; cationic polymers; and the like.

Examples of thickeners include gum arabic, carrageenan, gum karaya, gum tragacanth, gum carob, quince seed (quince), casein, dextrin, gelatin, sodium pectate, sodium alginate, methylcellulose, ethylcellulose, CMC, hydroxyethylcellulose, hydroxypropylcellulose, PVA, PVM, PVP, sodium polyacrylate, carboxyvinyl polymer, locust bean gum, guar gum, tamarind gum, dialkyl dimethyl ammonium cellulose sulfate, xanthan gum, magnesium aluminum silicate, bentonite, hectorite, A1 Mg silicate (bee gum), laponite, silicic anhydride, and the like.

Examples of sequestrants include 1-hydroxyethane-1,1-diphosphonic acid, 1-hydroxyethane-1,1-diphosphonic acid tetrasodium salt, disodium edetate, trisodium edetate, tetrasodium edetate, sodium citrate, sodium polyphosphate, sodium metaphosphate, gluconic acid, phosphoric acid, citric acid, ascorbic acid, succinic acid, edetic acid, trisodium ethylenediamine hydroxyethyl triacetate, and the like.

Examples of monosaccharides include trioses (e.g., D-glyceryl aldehyde and dihydroxyacetone); tetroses (e.g., D-erythrose, D-erythrulose, D-threose, and erythritol); pentoses (e.g., L-arabinose, D-xylose, L-lyxose, D-arabinose, D-ribose, D-ribulose, D-xylulose, and L-xylulose); hexoses (e.g., D-glucose, D-talose, D-psicose, D-galactose, D-fructose, L-galactose, L-mannose, and D-tagatose); heptoses (e.g., aldoheptose and heplose); octoses (e.g., octulose); deoxy sugars (e.g., 2-deoxy-D-ribose, 6-deoxy-L-galactose, and 6-deoxy-L-mannose); amino sugars (e.g., D-glucosamine, D-galactosamine, sialic acid, aminouronic acid, and muramic acid); uronic acids (e.g., D-glucuronic acid, D-mannuronic acid, L-guluronic acid, D-galacturonic acid, and L-iduronic acid); and the like.

Examples of oligosaccharides include sucrose, gentianose, umbelliferose, lactose, planteose, isolychnoses, α,α-trehalose, raffinose, lychnoses, umbilicin, stachyose verbascoses, and the like.

Examples of polysaccharides include cellulose, quince seed, chondroitin sulfate, starch, galactan, dermatan sulfate, glycogen, gum arabic, heparan sulfate, hyaluronic acid, gum tragacanth, keratan sulfate, chondroitin, xanthan gum, mucoitin sulfate, guar gum, dextran, keratosulfate, locust bean gum, succinoglycan, caronic acid, and the like.

Examples of amino acids include neutral amino acids (e.g., threonine and cysteine); basic amino acids (e.g., hydroxylysine); and the like. Examples of amino acid derivatives include sodium acyl sarcosinate (sodium lauroyl sarcosinate), acyl glutamate, sodium acyl β-alanine, glutathione, pyrrolidone carboxylic acid, and the like.

Examples of organic amines include monoethanolamine, diethanolamine, triethanolamine, morpholine, triisopropanolamine, 2-amino-2-methyl-1,3-propanediol, 2-amino-2-methyl-1-propanol, and the like.

Examples of polymer emulsions include acrylic resin emulsion, polyethyl acrylate emulsion, acrylic resin liquid, polyacrylalkyl ester emulsion, polyvinyl acetate resin emulsion, natural rubber latex, and the like.

Examples of pH adjusters include buffering agents, such as lactic acid-sodium lactate, citric acid-sodium citrate, and succinic acid-sodium succinate.

Examples of vitamins include vitamins A, B1, B2, B6, C, and E, and derivatives thereof, pantothenic acid and derivatives thereof, biotin, and the like.

Examples of antioxidants include tocopherols, dibutylhydroxytoluene, butylhydroxyanisole, gallic acid esters, and the like.

Examples of antioxidant aids include phosphoric acid, citric acid, ascorbic acid, maleic acid, malonic acid, succinic acid, fumaric acid, cephalin, hexametaphosphate, phytic acid, ethylenediaminetetraacetic acid, and the like.

Examples of other components that can be blended include preservatives (ethylparaben, butylparaben, chlorphenesin, phenoxyethanol, etc.); antiphlogistics (e.g., glycyrrhizic acid derivatives, glycyrrhetinic acid derivatives, salicylic acid derivatives, hinokitiol, zinc oxide, and allantoin); whitening agents (e.g., placenta extract, saxifrage extract, and arbutin); various extracts (e.g., phellodendron bark, coptis rhizome, lithospermum, peony, swertia herb, birch, sage, loquat, carrot, aloe, mallow, iris, grape, coix seed, sponge gourd, lily, saffron, cnidium rhizome, ginger, hypericum, restharrow, garlic, capsicum, citrus unshiu peel, Japanese angelica, and seaweed), activators (e.g., royal jelly, photosensitizers, and cholesterol derivatives); blood circulation promoters (e.g., nonanoic acid vanillylamide, nicotinic acid benzyl ester, nicotinic acid β-butoxyethyl ester, capsaicin, zingerone, cantharis tincture, ichthammol, tannic acid, α-borneol, tocopherol nicotinate, inositol hexanicotinate, cyclandelate, cinnarizine, tolazoline, acetylcholine, verapamil, cepharanthine, and γ-oryzanol); antiseborrheic agents (e.g., sulfur and thianthol); anti-inflammatory agents (e.g., tranexamic acid, thiotaurine, and hypotaurine); and the like.

Further, the following can also be blended, as appropriate: sequestering agents, such as disodium edetate, trisodium edetate, sodium citrate, sodium polyphosphate, sodium metaphosphate, gluconic acid, and malic acid; caffeine, tannin, verapamil, tranexamic acid and derivatives thereof; various crude drug extracts, such as licorice, Chinese quince, and Pyrola japonica; drugs, such as tocopherol acetate, glycyrrhetinic acid, glycyrrhizic acid, and derivatives thereof or salts thereof; whitening agents, such as vitamin C, magnesium ascorbyl phosphate, ascorbic acid glucoside, arbutin, and kojic acid; amino acids, such as arginine and lysine, and derivatives thereof; sugars, such as fructose, mannose, erythritol, trehalose, and xylitol; and the like.

Preferred embodiments of the oily solid composition according to the present invention will be described below.

First, in order to solidify an oily liquid medium with an aqueous solidifying agent, the present inventors examined aqueous liquid media compatible with both of them. The results are shown in Table 1.

The solidification properties were evaluated by visual observation, and the hardness was measured using a rheometer by reading the range when a probe with a diameter of 5.6 mm penetrated 2 mm at a speed of 20 mm/min.

The hardness of the oily solid composition of the present invention is preferably 10 to 500, and more preferably 20 to 250.

TABLE 1

| Test example | | 1-1 | 1-2 | 1-3 | 1-4 | 1-5 |
|---|---|---|---|---|---|---|
| Oily medium | Octyl methoxycinnamate | 10 | 10 | 10 | 10 | 10 |
| Aqueous | DPG | 10 | — | — | — | — |
| medium | PEG-8 | — | 10 | — | — | — |
| | Glycerol | — | — | 10 | — | — |
| | Propanediol | — | — | — | 10 | — |
| | Diglycerol | — | — | — | — | 10 |
| Oily medium ratio | | 0.5 | 0.5 | 0.5 | 0.5 | 0.5 |
| Compatibility (no solidifying agent added) | | Homogeneous | Homogeneous | Separation | Separation | Separation |
| | | phase | phase | | | |
| Solidifying agent | PEG-400 | 5 | 5 | 5 | 5 | 5 |
| Total | | 25 | 25 | 25 | 25 | 25 |
| Evaluation | Solidification properties | Gelation | Gelation | Separation | Separation | Separation |
| | (immediately after) | | | | | |
| | Solidification properties | Gelation | Gelation | Separation | Separation | Separation |
| | (1 day after) | | | | | |
| | Hardness (1 day after) | 365 | 350 | — | — | — |

As shown in Table 1, first, ethylhexyl methoxycinnamate, which is a relatively high-polar oil content, was used as an oily liquid medium, and aqueous liquid media compatible with ethylhexyl methoxycinnamate at a mass ratio of 1:1 were selected.

As a result, dipropylene glycol (Test Example 1-1) and polyethylene glycol 400 (Test Example 1-2) were compatible with the ethylhexyl methoxycinnamate at a mass ratio of 1:1; however, glycerol, propanediol, and diglycerol (Test Examples 1-3 to 1-5) were incompatible and separated.

Further, when polyethylene glycol-400 was added as an aqueous solidifying agent to attempt solidification, gelation of the entire system was observed in Test Examples 1-1 and 1-2.

Next, the present inventors examined oily liquid media. The results are shown in Table 2.

TABLE 2

| Test example | | 2-1 | 2-2 | 2-3 | 2-4 | 2-5 |
|---|---|---|---|---|---|---|
| Oily medium | Octyl salicylate | 10 | 10 | — | — | — |
| | Dimethicone (6CS) | — | — | 10 | — | — |
| | Isopropyl myristate | — | — | — | 10 | — |
| | Phenyl silicone | — | — | — | — | 10 |
| Aqueous | DPG | 10 | — | 10 | 10 | 10 |
| medium | PEG-8 | — | 10 | — | — | — |
| Oily medium ratio | | 0.5 | 0.5 | 0.5 | 0.5 | 0.5 |
| Compatibility (no solidifying agent added) | | Homogeneous | Separation | Separation | Separation | Separation |
| | | phase | | | | |
| Solidifying agent | PEG-400 | 5 | 5 | 5 | 5 | 5 |
| Total | | 25 | 25 | 25 | 32 | 32 |
| Evaluation | Solidification properties | Gelation | Separation | Separation | Separation | Separation |
| | (immediately after) | | | | | |
| | Solidification properties | Gelation | Separation | Separation | Separation | Separation |
| | (1 day after) | | | | | |
| | Hardness (1 day after) | 345 | — | — | — | — |

As shown in Table 2, ethylhexyl salicylate formed a homogeneous phase when combined with dipropylene glycol (Test Example 2-1); however, good compatibility with PEG-8 was not observed (Test Example 2-2). Further, low-polar silicone oil, isopropyl myristate, and phenyl silicone did not show good compatibility with either DPG or PEG-8 (Test Examples 2-3 to 2-5).

Next, the present inventors examined solidifying agents. The results are shown in Table 3.

TABLE 3

| Test example | | 3-1 | 3-2 | 3-3 | 3-4 | 3-5 |
|---|---|---|---|---|---|---|
| Oily medium | Ethylhexyl | 7.5 | 7.5 | 7.5 | 7.5 | 7.5 |
| | methoxycinnamate | | | | | |
| | Ethylhexyl salicylate | 5 | 5 | 5 | 5 | 5 |
| Aqueous | DPG | 9 | 9 | 9 | 9 | 9 |
| medium | PEG-8 | 5.5 | 5.5 | 5.5 | 5.5 | 5.5 |
| Oily medium ratio | | 0.46 | 0.46 | 0.46 | 0.46 | 0.46 |
| Compatibility (no solidifying agent added) | | Homogeneous | Homogeneous | Homogeneous | Homogeneous | Homogeneous |
| | | phase | phase | phase | phase | phase |
| Solidifying agent | PEG-400 | 5 | — | — | — | — |
| | Bis-PEG-18 methyl ether | — | 10 | — | — | — |
| | dimethylsilane | | | | | |
| | Polyethylene glycol | — | — | 2 | — | — |
| | decyltetradeceth-20 | | | | | |
| | hexamethylene | | | | | |
| | diisocyanate copolymer | — | — | — | 1 | — |
| | Hydroxypropyl | | | | | |
| | methylcellulose stearoxy | | | | | |
| | ether | | | | | |
| | 12-Hydroxystearic acid | — | — | — | — | 2 |
| Evaluation | Solidification properties | Gelation | Gelation | Sol | Precipitation | Sol |
| | (immediately after) | | | | | |
| | Solidification properties | Gelation | Gelation | Gelation | Precipitation | Week gelation |
| | (1 day after) | | | | | |
| | Hardness (1 day after) | 195 | 85 | 20 | — | — |

As shown in Table 3, the oily medium used was a mixed solvent of ethylhexyl methoxycinnamate and ethylhexyl salicylate, and the aqueous medium used was a mixed solvent of DPG and PEG-8. Both showed good compatibility.

PEG-400, bis-PEG-18 methyl ether dimethylsilane, and PEG-240·decyltetradeceth-20·hexamethylene diisocyanate copolymer, which are aqueous solidifying agents, had gelling ability for the aqueous medium/oily medium mixed system; however, 12-hydroxystearic acid, which is an oily solidifying agent, alone showed only very weak solidification ability.

Further, the present inventors examined the polarity of solidifying agents and their solidification ability. The results are shown in Table 4.

TABLE 4

| Test example | | | 4-1 | 4-2 | 4-3 | 4-4 | 4-5 | 4-6 | 4-7 |
|---|---|---|---|---|---|---|---|---|---|
| Oily medium | | Ethylhexyl | 22.5 | 22.5 | 22.5 | 22.5 | 22.5 | 22.5 | 22.5 |
| | | methoxycinnamate | | | | | | | |
| | | Ethylhexyl salicylate | 5 | 5 | 5 | 5 | 5 | 5 | 5 |
| Aqueous | | DPG | 27 | 27 | 27 | 27 | 27 | 27 | 27 |
| medium | | PEG-8 | 20.5 | 20.5 | 20.5 | 20.5 | 20.5 | 20.5 | 20.5 |
| Oily medium ratio | | | 0.32 | 0.32 | 0.32 | 0.32 | 0.32 | 0.32 | 0.32 |
| High | Aqueous | Gellan gum (sugar derivative) | 15 | — | — | — | — | — | — |
| polarity | | PEG-400 | — | 15 | — | — | — | — | — |
| ↑ | | Polyethylene glycol | — | — | 15 | — | — | — | — |
| solidifying | | decyltetradeceth-20 | | | | | | | |
| agent | | hexamethylene diisocyanate | | | | | | | |
| ↓ | | copolymer | | | | | | | |
| low | Oily | 12-Hydroxystearic acid | — | — | — | 15 | — | — | — |
| polarity | | Dibutyl lauryl glutamide | — | — | — | — | 15 | — | — |
| | | Dextrin palmitate | — | — | — | — | — | 15 | — |
| | | Microcrystalline wax | — | — | — | — | — | — | 15 |

| | | | | | | | | |
|---|---|---|---|---|---|---|---|---|
| Evaluation | Solidification properties | x | Gelation | Weak | Gelation | Gelation | x | x |

gelation

As is clear from Table 4, PEG-400, which is an aqueous solidifying agent, and 12-hydroxystearic acid and dibutyl lauroyl glutamide, which are oily solidifying agents, had gelling ability for the aqueous medium/oily medium mixed system; however, ADEKA NOL, which is an aqueous solidifying agent, had only weak solidification ability, and gellan gum (sugar derivative), which is a highly-polar aqueous solidifying agent, and dextrin palmitate and microcrystalline wax, which are low-polar oily solidifying agents, did not have solidification ability.

Further, as for various aqueous liquid media, the present inventors used ethylhexyl methoxycinnamate, which is a relatively high-polar oil content, as an oily liquid medium, and selected aqueous liquid media compatible with the ethylhexyl methoxycinnamate at a mass ratio of 1:1. The results are shown in Table 5.

TABLE 5

| | Ethylhexyl |
|---|---|
| | methoxycinnamate |
| PEG-6 | ◯ |
| PEG-8 | ◯ |
| 1,8-BG | X |
| 1,3-PD | X |
| DPG | ◯ |
| DG | X |
| DDG | X |
| IPG | X |
| PG | X |
| 1,2HD | ◯ |
| PEG/PPG-14/7 | ◯ |
| dimethyl ether | |
| PEG/PPG-9/2 | ◯ |
| dimethyl ether | |
| PEG/PPG-36/41 | ◯ |
| dimethyl ether | |
| PEG/PPG-55/28 | ◯ |
| dimethyl ether | |
| PEG/PPG-17/4 | ◯ |
| dimethyl ether | |

As is clear from Table 5, it is understood that dipropylene glycol (DPG), polyethylene glycols that are liquid at room temperature (PG; PEG-6 and PEG-8), 1,2-hexanediol (1,2-HD), alkylene oxide derivatives (PEG/PPG-14/7 dimethyl ether, PEG/PPG-9/2 dimethyl ether, PEG/PPG-36/41 dimethyl ether, PEG/PPG-55/28 dimethyl ether, and PEG/PPG-17/4 dimethyl ether) can serve as excellent aqueous liquid media for the oily liquid medium (ethylhexyl methoxycinnamate; MCX).

Further, the present inventors selected aqueous liquid media compatible with ethylhexyl methoxycinnamate at various mass ratios. The results of the examination are shown in Table 6.

TABLE 6

| | Oily medium | | | | | | | | | | | |
|---|---|---|---|---|---|---|---|---|---|---|---|---|
| | Ethylhexyl methoxycinnamate | | | | | | | | | | | |
| Concentration | 90 | 66.6 | 50 | 33.3 | 30 | 25 | 20 | 16.7 | 14.3 | 12.5 | 11.5 | 10 |
| PEG/PPG-36/41 | ∘ | ∘ | ∘ | ∘ | ∘ | ∘ | ∘ | ∘ | ∘ | ∘ | ∘ | ∘ |
| dimethyl ether | | | | | | | | | | | | |
| PEG/PPG-55/28 | ∘ | ∘ | ∘ | ∘ | ∘ | ∘ | ∘ | ∘ | ∘ | ∘ | ∘ | ∘ |
| dimethyl ether | | | | | | | | | | | | |
| PEG/PPG-14/7 | ∘ | ∘ | ∘ | ∘ | ∘ | ∘ | ∘ | ∘ | ∘ | ∘ | ∘ | ∘ |
| dimethyl ether | | | | | | | | | | | | |
| PEG/PPG-17/4 | ∘ | ∘ | ∘ | ∘ | ∘ | ∘ | ∘ | ∘ | ∘ | ∘ | ∘ | ∘ |
| dimethyl ether | | | | | | | | | | | | |
| PEG/PPG-9/2 | ∘ | ∘ | ∘ | ∘ | ∘ | ∘ | ∘ | ∘ | ∘ | ∘ | ∘ | ∘ |
| dimethyl ether | | | | | | | | | | | | |
| PEG-6 | ∘ | ∘ | ∘ | ∘ | ∘ | ∘ | ∘ | ∘ | ∘ | ∘ | ∘ | ∘ |
| PEG-8 | ∘ | ∘ | ∘ | ∘ | ∘ | ∘ | ∘ | ∘ | ∘ | ∘ | ∘ | ∘ |
| DPG | ∘ | ∘ | ∘ | ∘ | ∘ | ∘ | ∘ | ∘ | ∘ | ∘ | ∘ | ∘ |
| IPG | x | x | x | x | x | x | ∘ | ∘ | ∘ | ∘ | ∘ | ∘ |
| PG | x | x | x | x | x | x | x | ∘ | ∘ | ∘ | ∘ | ∘ |
| 1,3-BG | x | x | x | x | x | x | x | x | ∘ | ∘ | ∘ | ∘ |
| 1,3-PD | x | x | x | x | x | x | x | x | x | x | x | x |
| DG | x | x | x | x | x | x | x | x | x | x | x | x |
| DDG | x | x | x | x | x | x | x | x | x | x | x | x |

As is clear from Table 6, the good solvents showing high compatibility in Table 5 are compatible with the oily liquid medium at any mass ratio. In contrast, isopropylene glycol (IPG), propylene glycol (PG), and 1,3-butylene glycol, which are highly hydrophilic solvents, were made compatible by changing the ratio; however, 1,3-propanediol (1,3-PD), diglycerol (DG), and dynamite glycerol (DDG), which are extremely highly hydrophilic solvents, were not compatible even after changing the ratio.

In addition, the present inventors examined the behavior of aqueous liquid media that could hardly or never be solvents for the oily liquid medium by themselves, specifically 1,3-butylene glycol (1,3-BG), diglycerol (DG), 1,3-propanediol (1,3-PD), propylene glycol (PG), isopropylene glycol (IPG), and dynamite glycerol (DDG), in coexistence with dipropylene glycol, which is a good solvent. The results are shown in Table 7.

TABLE 7

| | Oily medium + DPG | | | | | | | | | | |
|---|---|---|---|---|---|---|---|---|---|---|---|
| | Ethylhexyl | | | Ethylhexyl | | | Ethylhexyl | Ethylhexyl | | | |
| | methoxycinnamate | | | methoxycinnamate:DPG = 1:1 | | | methoxycinnamate:DPG = 2:1 | methoxycinnamate:DPG = 1:2 | | | |
| Oily medium + DPG:poor solvent | 1:2 | 1:1 | 2:1 | 1:2 | 1:1 | 2:1 | 3:1 | 3:1 | 4:1 | 6:1 | 9:1 |
| 1,3-BG | x | x | x | x | x | x | x | ∘ | ∘ | ∘ | ∘ |
| DG | x | x | x | x | x | x | x | x | x | x | ∘ |
| 1,3-PD | x | x | x | x | x | x | x | x | x | x | ∘ |
| PG | x | x | x | x | x | x | x | ∘ | ∘ | ∘ | ∘ |
| IPG | x | x | x | x | x | ∘ | Δ | ∘ | ∘ | ∘ | ∘ |
| DDG | x | x | x | x | x | x | x | x | x | x | ∘ |

Table 7 indicates that the compatibility of the aqueous liquid media with low affinity with the oily liquid medium with the oily liquid medium is improved due to the coexistence of an aqueous liquid medium with high affinity (dipropylene glycol), revealing that 1,3-propanediol (1,3-PD), diglycerol (PG), and dynamite glycerol (DDG) are also compatible as aqueous liquid solvents depending on the coexisting ratio.

The present inventors further examined ethylhexyl methoxycinnamate:DPG=1:2, at which the compatibility was improved in Table 6. The results are shown in Table 8.

TABLE 8

| | Oily medium + DPG | | | | | | | | | | | |
|---|---|---|---|---|---|---|---|---|---|---|---|---|
| | Ethylhexyl methoxycinnamate: DPG = 1:2 | | | | | | | | | | | |
| Concentration | 90 | 66.6 | 50 | 33.3 | 30 | 25 | 20 | 16.7 | 14.3 | 12.5 | 11.1 | 10 |
| PEG/PPG-36/41 | ∘ | ∘ | ∘ | ∘ | ∘ | ∘ | ∘ | ∘ | ∘ | ∘ | ∘ | ∘ |
| dimethyl ether | | | | | | | | | | | | |
| PEG/PPG-55/28 | ∘ | ∘ | ∘ | ∘ | ∘ | ∘ | ∘ | ∘ | ∘ | ∘ | ∘ | ∘ |
| dimethyl ether | | | | | | | | | | | | |
| PEG/PPG-14/7 | ∘ | ∘ | ∘ | ∘ | ∘ | ∘ | ∘ | ∘ | ∘ | ∘ | ∘ | ∘ |
| dimethyl ether | | | | | | | | | | | | |
| PEG/PPG-17/4 | ∘ | ∘ | ∘ | ∘ | ∘ | ∘ | ∘ | ∘ | ∘ | ∘ | ∘ | ∘ |
| dimethyl ether | | | | | | | | | | | | |
| PEG/PPG-9/2 | ∘ | ∘ | ∘ | ∘ | ∘ | ∘ | ∘ | ∘ | ∘ | ∘ | ∘ | ∘ |
| dimethyl ether | | | | | | | | | | | | |
| PEG-6 | ∘ | ∘ | ∘ | ∘ | ∘ | ∘ | ∘ | ∘ | ∘ | ∘ | ∘ | ∘ |
| PEG-8 | ∘ | ∘ | ∘ | ∘ | ∘ | ∘ | ∘ | ∘ | ∘ | ∘ | ∘ | ∘ |
| DPG | ∘ | ∘ | ∘ | ∘ | ∘ | ∘ | ∘ | ∘ | ∘ | ∘ | ∘ | ∘ |
| IPG | ∘ | ∘ | ∘ | ∘ | ∘ | ∘ | ∘ | ∘ | ∘ | ∘ | ∘ | ∘ |
| PG | x | x | x | x | ∘ | ∘ | ∘ | ∘ | ∘ | ∘ | ∘ | ∘ |
| 1,3-BG | x | x | x | x | x | ∘ | ∘ | ∘ | ∘ | ∘ | ∘ | ∘ |
| 1,3-PD | x | x | x | x | x | x | x | x | x | ∘ | ∘ | ∘ |
| DG | x | x | x | x | x | x | x | x | x | x | ∘ | ∘ |
| DDG | x | x | x | x | x | x | x | x | x | x | x | ∘ |

From Table 8, it was revealed that by containing an oily liquid medium and an aqueous liquid medium with high affinity at 1:2, the compatibility with the aqueous medium was improved. 1,3-Propanediol, diglycerol, and dynamite glycerol, which were incompatible at any concentration in Table 6, were also compatible when mixed with ethylhexyl methoxycinnamate and DPG at 1:2, depending on the concentration.

The present inventors examined the state of solid compositions when an oil-absorbing powder was contained. The results are shown in Table 9.

The smoothness and stability were evaluated as shown below.

### [Method for Evaluating Smoothness]

A panel of 10 experts applied the solid composition to the skin, and evaluation was performed based on the number of experts who could feel smoothness.
⊚: 8 or more out of 10 experts felt smoothness.
◯: 7 out of 10 experts felt smoothness.
◯Δ: 6 out of 10 experts felt smoothness.
Δ: 4 or 5 out of 10 experts felt smoothness.
X: 3 or fewer out of 10 experts felt smoothness.

### [Method for Evaluating Stability]

It was visually evaluated whether separation, lump formation, and the like occurred at 25° C. with a humidity of 50%.
◯: Separation or lump formation did not occur.
X: Separation and lump formation occurred.

TABLE 9

| | Test example | 9-1 | 9-2 | 9-3 | 9-4 | 9-5 | 9-6 | 9-7 | 9-8 | 9-9 | 9-10 | 9-11 | 9-12 |
|---|---|---|---|---|---|---|---|---|---|---|---|---|---|
| Extender | Talc | 15.3 | 8.28 | 8.28 | 8.28 | | | | | | | | |
| pigment | Mica | | | | | 8.28 | 8.28 | 8.28 | 8.28 | 3.28 | 0.28 | 0.28 | 6.78 |
| | Silica-coated mica | | | | | | 5 | 5 | 5 | 5 | 5 | 5 | 5 |
| | Synthetic [symbol] | 5 | 5 | 5 | 5 | 5 | 5 | 5 | 5 | 5 | 5 | 5 | 5 |
| | Plate barium sulfate | | | | | | | | | | | | 1 |
| | Boron nitride | 3.5 | 3.5 | 3.5 | 3.5 | 3.5 | 3.5 | 3.5 | 3.5 | 3.5 | 3.5 | 3.5 | 3.5 |
| Fine titanium | Hydrophobized fine | 9-1 | | | | | | | | | | | |
| oxide | titanium oxide | | | | | | | | | | | | |
| Pigment | Hydrophobized | 7 | 7 | 7 | 7 | 7 | 7 | 7 | 7 | 7 | 7 | 7 | 7 |
| | pigment-grade | | | | | | | | | | | | |
| | titanium oxide | | | | | | | | | | | | |
| | Hydrophobized iron | 0.1 | 0.1 | 0.1 | 0.1 | 0.1 | 0.1 | 0.1 | 0.1 | 0.1 | 0.1 | 0.1 | 0.1 |
| | oxide red | | | | | | | | | | | | |
| | Hydrophobized iron | 0.9 | 0.9 | 0.9 | 0.9 | 0.9 | 0.9 | 0.9 | 0.9 | 0.9 | 0.9 | 0.9 | 0.9 |
| | oxide yellow | | | | | | | | | | | | |
| | Hydrophobized iron | 0.07 | 0.07 | 0.07 | 0.07 | 0.07 | 0.07 | 0.07 | 0.07 | 0.07 | 0.07 | 0.07 | 0.07 |
| | oxide black | | | | | | | | | | | | |
| Spherical | Silica | 15 | 15 | 15 | 15 | 15 | 15 | 15 | 15 | 15 | 13 | 13 | 15 |
| powder | | | | | | | | | | | | | |
| Oil-absorbing | Porous high-oil- | | | | | | | | | | | 5 | |
| powder | absorbing silica | | | | | | | | | | | | |
| | (Dimethicone/vinyl | | 5 | 5 | 5 | 5 | 5 | 5 | 5 | 5 | 5 | | 5.5 |
| | dimethicone) | | | | | | | | | | | | |
| | crosspolymer | | | | | | | | | | | | |
| Preservative | [symbol] | 0.15 | 0.15 | 0.15 | 0.15 | 0.15 | 0.15 | 0.15 | 0.15 | 0.15 | 0.15 | 0.15 | 0.15 |
| Total amount of powders (%) | | 50 | 45 | 45 | 45 | 50 | 50 | 50 | 50 | 45 | 40 | 40 | 50 |
| Absorbed oil | Dimethicone ( [symbol] ) | | 17 | 18 | 18 | 18.5 | 18.5 | 18.5 | 18.5 | 18.5 | 18.5 | 18.5 | 22 |
| | Bis-butyl dimethicone | | 1 | 1 | 1 | 0.5 | 0.5 | 0.5 | 0.5 | 0.5 | 0.5 | 0.5 | 1 |
| | polyglyceryl-3 | | | | | | | | | | | | |
| Oil- [symbol] liquid | [symbol] | 0.5 | | | | | | | | | | | |
| | [symbol] | 7.5 | 5 | 5 | 5 | 5 | 5 | 5 | 5 | 7.5 | 7.5 | 7.5 | 7.5 |
| | methoxycinnamate | | | | | | | | | | | | |
| | [symbol] | 5 | 5 | 5 | 5 | 5 | 5 | 5 | 5 | 5 | 5 | 5 | 3.5 |
| | Dipropylene glycol | 9 | 9 | 9 | 9 | 7 | 7 | 7 | 7 | 8 | 9 | 9 | 8 |
| Water-soluble | PEG- [symbol] | | 5 | 5 | 5 | 9 | 10 | 11 | 12 | 13 | 16.5 | 16.5 | |
| liquid | | | | | | | | | | | | | |
| | PEG- [symbol] | 10 | 10 | 10 | 10 | | | | | | | | |
| | 1,2-Hexeradiol | | | | | | | | | | | | 3 |
| | [symbol] | | | | | | | | | | | | 2 |
| | PEG/PPG-14/7 | | | | | | | | | | | | 0.1 |
| | dimethyl ether | | | | | | | | | | | | |
| Oil-soluble | (PEG-240/ | | | | 1 | | | | | | | | |
| solid | decyltetrodoneth- | | | | | | | | | | | | |
| | 20/H [symbol] ) | | | | | | | | | | | | |
| | copolymer | | | 1 | 1 | | | | | | | | |
| | Hydroxypropyl cellulose | | | | | | | | | | | | |
| Water-soluble | Bis-PEG-18 methyl | 9 | | | | | | | | | | | |
| solid | ether dimeth [symbol] | | | | | | | | | | | | |
| | PEG-400 | 3 | 3 | 1 | | 5 | 4 | 3 | 2 | 2.5 | 3 | 3 | 2.5 |
| Total amount of lipids (%) | | 50 | 55 | 55 | 55 | 50 | 50 | 50 | 50 | 55 | 60 | 60 | 50 |
| | Hardness | 308 | 218 | [symbol] | 170 | 720 | 580 | 480 | 303 | 159 | 42 | 150 | 27 |
| | Smoothness | ∘ | ⊚ | ⊚ | ∘Δ | x | Δ | ∘Δ | ∘ | ⊚ | ⊚ | ∘ | ⊚ |
| | Stability | ∘ | ∘ | ∘ | ∘ | ∘ | ∘ | ∘ | ∘ | ∘ | ∘ | ∘ | ∘ |

[symbol]

indicates data missing or illegible when filed

From Table 9, when an oil-absorbing powder is blended, the smoothness tends to be improved; however, it is preferable that the total amount of liquids is slightly larger (55% by mass or more).

Formulation examples of the cosmetic according to the present invention are shown below.

Formulation Example 1: Skin care solid

| | Blending |
|---|---|
| | amount |
| Components | (% by mass) |
| Ethylhexyl methoxycinnamate | 15% |
| Ethylhexyl salicylate | 10% |
| Dipropylene glycol | 21% |
| PEG-6 | 26% |
| Bis-PEG-18 methyl ether dimethylsilane | 20% |
| PEG-400 | 8% |

Formulation Example 2: Lip stick

| | Blending |
|---|---|
| | amount |
| Components | (% by mass) |
| Ethylhexyl methoxycinnamate | 15% |
| Ethylhexyl salicylate | 10% |
| Dipropylene glycol | 21% |
| PEG-6 | 23% |
| Bis-PEG-18 methyl ether dimethylsilane | 20% |
| PEG-400 | 8% |
| Red No. 202 (INCI Name: | 3% |
| CI 15850, Red 7) | |

Formulation Example 3: Foundation

| | Blending |
|---|---|
| | amount |
| Components | (% by mass) |
| Mica | Up to 100 |
| Silica-coated mica | 5% |
| Boron nitride | 3.5% |
| Hydrophobized pigment-grade | 7% |
| titanium oxide | |
| Hydrophobized iron oxide red | 0.1% |
| Hydrophobized iron oxide yellow | 0.9% |
| Hydrophobized iron oxide black | 0.07% |
| Silica | 15% |
| (Dimethicone/vinyl dimethicone) | 5% |
| crosspolymer | |
| Clofentezine | 0.15% |
| Dimethicone (10 CS) | 18.5% |
| Bis-butyl dimethicone polyglyceryl-3 | 0.5% |
| Ethylhexyl methoxycinnamate | 7.5% |
| Ethylhexyl salicylate | 5% |
| Dipropylene glycol | 8% |
| PEG-6 | 13% |
| PEG-400 | 2.5% |

Formulation Example 4: Eyeshadow

| | Blending |
|---|---|
| | amount |
| Components | (% by mass) |
| Mica | Up to 100 |
| Silica-coated mica | 9% |
| Talc | 5% |
| Boron nitride | 3.5% |
| Pearl agent(Mica/iron oxide) | 12% |
| Silica | 14% |
| (Dimethicone/vinyl dimethicone) | 6% |
| crosspolymer | |
| Clofentezine | 0.15% |
| Dimethicone (100CS) | 18.5% |
| Bis-butyl dimethicone polyglyceryl-3 | 0.5% |
| Ethylhexyl methoxycinnamate | 5% |
| Ethylhexyl salicylate | 5% |
| Dipropylene glycol | 7% |
| PEG-300 | 10% |
| PEG-400 | 1.5% |

Formulation Example 5: Cleaning agent

| | Blending |
|---|---|
| | amount |
| Components | (% by mass) |
| Ethylhexyl methoxycinnamate | 15% |
| Ethylhexyl salicylate | 10% |
| Dipropylene glycol | 19% |
| PEG-6 | 26% |
| Bis-PEG-18 methyl ether dimethylsilane | 20% |
| PEG-400 | 8% |
| Lauryl Betaine | 2% |

Formulation Example 6: Cleaning agent

| | Blending |
|---|---|
| | amount |
| Components | (% by mass) |
| Ethylhexyl methoxycinnamate | 15% |
| Ethylhexyl salicylate | 10% |
| Dipropylene glycol | 18% |
| PEG-6 | 26% |
| Bis-PEG-18 methyl ether dimethylsilane | 20% |
| PEG-400 | 8% |
| Triglyceryl-6 dicaprate | 3% |

What is claimed is:

1. A solid composition comprising:
an oily liquid medium that separates when mixed with water at 1:1;
an aqueous liquid medium that is an organic liquid medium other than water, that forms a homogeneous phase when mixed with water at 1:1, and that forms a homogeneous phase when mixed with the oily liquid medium; and
an aqueous solidifying agent that is soluble in the aqueous liquid medium and that gels and/or solidifies the aqueous liquid medium, and/or an oily solidifying agent that is soluble in the oily liquid medium and that gels and/or solidifies the oily liquid medium,
wherein the oily liquid medium and aqueous liquid medium are gelled and/or solidified with the aqueous solidifying agent and/or oily solidifying agent, and
the total amount of the aqueous liquid medium, oily liquid medium, aqueous solidifying agent, and oily solidifying agent is 90% by mass or more of the amount of the composition excluding powder.

2. The solid composition according to claim 1, comprising an oil-absorbing powder containing an oil content other than the oily liquid medium.

3. The solid composition according to claim 1 or 2, having a hardness of 10 to 500 when a probe with a diameter of 5.6 mm penetrates at 20 mm/min using a rheometer.

4. The solid composition according to any one of claims 1 to 3, wherein:
the oily liquid medium is one or two or more selected from the group consisting of, as phenyl-containing substances, cinnamic acid derivatives selected from ethylhexyl methoxycinnamate, isopropyl methoxycinnamate, and isoamyl methoxycinnamate; PABA derivatives selected from para-aminobenzoic acid, ethyl PABA, ethyl-dihydroxypropyl PABA, ethylhexyl-dimethyl PABA, and glyceryl PABA; salicylic acid derivatives selected from homosalate, ethylhexyl salicylate, dipropylene glycol salicylate, and TEA salicylate; benzophenone derivatives selected from benzophenone-1, benzophenone-2, benzophenone-3 or oxybenzone, benzophenone-4, benzophenone-5, benzophenone-6, benzophenone-8, benzophenone-9, and benzophenone-12; benzylidene camphor derivatives selected from 3-benzylidene camphor, 4-methylbenzylidene camphor, benzylidene camphor sulfonic acid, benzalkonium methosulfate camphor, terephthalylidene dicamphor sulfonic acid, and polyacrylamide methylbenzylidene camphor; triazine derivatives selected from anisotriazine, ethylhexyltriazone, diethylhexylbutamidotriazone, 2,4,6-tris(diisobutyl-4′-aminobenzalmalonate)-s-triazine, 2,4-bis-[{4-(2-ethylhexyloxy)-2-hydroxy}-phenyl]-6-(4-methoxyphenyl)-1,3,5-triazine, and 2,4,6-tris [4-(2-ethylhexyloxycarbonyl) anilino]1,3,5-triazine; phenylbenzimidazole derivatives selected from disodium phenyl dibenzimidazole tetrasulfonate; phenylbenzotriazole derivatives selected from drometrizole trisiloxane and methylene bis(benzotriazolyl tetramethylbutylphenol); anthranil derivatives selected from menthyl anthranilate; imidazoline derivatives selected from ethylhexyl dimethoxy benzylidene dioxoimidazoline propionate; benzalmalonate derivatives selected from polyorganosiloxanes with benzalmalonate functional groups; 4,4-diarylbutadiene derivatives selected from 1,1-dicarboxy (2,2′-dimethylpropyl)-4,4-diphenylbutadiene; hexyl diethylaminohydroxybenzoylbenzoate, and bis-ethylhexyloxyphenol methoxyphenyl triazine; and diols and triols that are incompatible with water at 1:1;
the aqueous liquid medium is one or two or more selected from the group consisting of dipropylene glycol, polyethylene glycols that are liquid at room temperature, PEG/PPG-14/7 dimethyl ether, PEG/PPG-9/2 dimethyl ether, PEG/PPG-36/41 dimethyl ether, PEG/PPG-55/28 dimethyl ether, PEG/PPG-17/4 dimethyl ether, 1,2-hexanediol, isopropylene glycol, propylene glycol, and 1,3-butylene glycol;
the aqueous solidifying agent is one or two or more selected from the group consisting of bis-polyethylene glycol methyl ether dimethylsilane, polyethylene glycol·decyltetradeceth-20·hexamethylene diisocyanate copolymer, polyethylene glycols that are solid at 25° C., and polyurethane-59; and
the oily solidifying agent is one or two or more selected from the group consisting of dibutyl lauroyl glutamide, dibutyl ethylhexanoyl glutamide, polyamide-3, polyamide-8, and 12-hydroxystearic acid.
